SEQUENCE LISTING (4 sequences)

SEQ ID NO: 1 - DNA, 23 bp, Artificial
  oligonucleotides
aattagatgtcatacagaccaag

SEQ ID NO: 2 - DNA, 119 bp, Hepatitis B virus
agtgtggattcgcactcctccagcctatagaccaccaaatgcccctatcttatcaacacttccggaaactactgttgttagacgacgggaccgaggcaggtcccctagaagaagaactc

SEQ ID NO: 3 - DNA, 19 bp, Artificial
  forward primer
agtgtggattcgcactcct

SEQ ID NO: 4 - DNA, 23 bp, Artificial
  reverse primer
gagttcttcttctaggggacctg

(19) United States
(12) Patent Application Publication
(10) Pub. No.: US 2006/0118417 A1
(43) Pub. Date: Jun. 8, 2006

(54) Nucleic acid purification method using hydrogen bonding and electric field

(76) Inventors: Young-a Kim, Gyeonggi-do (KR); Jun-hong Min, Gyeonggi-do (KR); Kui-hyun Kim, Daejeon-si (KR); Myo-yong Lee, Gyeonggi-do (KR); Su-hyeon Kim, Seoul (KR); Young-nam Kwon, Gyeonggi-do (KR); Jeong-gun Lee, Seoul (KR); Joon-ho Kim, Gyeonggi-do (KR)

Correspondence Address: CANTOR COLBURN, LLP, 55 GRIFFIN ROAD SOUTH, BLOOMFIELD, CT, 06002, US

(21) Appl. No.: 11/281,226

(22) Filed: Nov. 16, 2005

(30) Foreign Application Priority Data
Dec. 6, 2004 (KR) 10-2004-0101657

(51) Int. Cl. C07H 21/04

(52) U.S. Cl. 204/450; 536/25.4

(57) ABSTRACT

Provided is a method of purifying nucleic acids using hydrogen bonding and an electric field, including: bringing a sample containing target nucleic acids into contact with an electrode coated with a material capable of forming hydrogen bonds with the target nucleic acids; applying a positive voltage to the electrode to move the target nucleic acids closer to the electrode so as to form hydrogen bonds with the material on the electrode; washing the electrode; and applying to the electrode a negative voltage to elute the bound target nucleic acids. According to the method, selectivity to nucleic acids and proteins increases due to hydrogen bonding, nucleic acid purification is possible within a short time through an electric field, and the bound nucleic acids can be efficiently eluted.

### BACKGROUND OF THE INVENTION

This application claims the benefit of Korean Patent Application No. 10-2004-0101657, filed on Dec. 6, 2004, in the Korean Intellectual Property Office, the disclosure of which is incorporated herein in its entirety by reference.

1. Field of the Invention

The present invention relates to a method of purifying nucleic acids using hydrogen bonding and an electric field.

2. Description of the Related Art

Isolation methods of DNA from cells were performed using materials that have the proclivity of binding to DNA. Examples of materials for isolation methods of DNA are silica, glass fiber, anion exchange resin and magnetic beads (Rudi, K. et al., Biotechniqures 22, 506-511 (1997); and Deggerdal, A. et al., Biotechniqures 22, 554-557 (1997)). To avoid manual steps and to remove operator errors, several automatic machines were developed for high-throughput DNA extraction.

Conventionally, a method of purifying nucleic acids using a solid phase was known. For example, U.S. Pat. No. 5,234,809 discloses a method of purifying nucleic acids using a solid phase to which nucleic acids are bound. Specifically, the method includes mixing a starting material, a chaotropic material and a nucleic acid binding solid phase; separating the solid phase with the nucleic acid bound thereto from the liquid; and washing the solid phase nucleic acid complexes. However, this method is time consuming and complicated, and thus is not suitable for a Lab-On-a-Chip (LOC).

The method also has a problem of the use of the chaotropic material. That is, when the chaotropic material is not used, nucleic acids are not bound to the solid phase. The chaotropic material is toxic to humans, and thus should be handled with extreme caution. Also, the chaotropic material acts as an inhibitor in the subsequent step, and thus should be removed from nucleic acids during or after purification.

U.S. Pat. No. 6,291,166 discloses a method of archiving nucleic acids using a solid phase matrix. This method is advantageous in that since nucleic acids are irreversibly bound to the solid phase matrix, a delayed analysis or repeated analysis for the nucleic acid solid phase matrix complexes is possible. However, according to this method, Al, which has a positively-charged surface should be rendered hydrophilic with basic materials, such as NaOH, and nucleic acids are irreversibly bound to the Al rendered hydrophilic, and thus cannot be separated from Al. Thus, Al cannot be used to purify nucleic acids. In addition, the solid phase matrix does not have distinct selectivity to DNA and proteins.

WO 97/41219 discloses a method of purifying nucleic acids using an electric field, including: exposing an electrode to a mixture containing nucleic acids; applying to said electrode a nucleic acid attracting voltage; and removing said electrode from said mixture. In this method, an electric field is used to attract nucleic acids, but the coating of nucleic acid monomers on an electrode and hydrogen bonding are not used.

U.S. Pat. No. 6,518,022 discloses a method of enhancing hybridization efficiency between target nucleic acid and probe by applying electric field. In this method, the hybridization efficiency is enhanced using an electric field, but the coating of nucleic acid monomers on an electrode and hydrogen bonding are not used.

To perform an efficient polymerase chain reaction (PCR) for LOC implementation, a purification process of nucleic acids is required after cell lysis. However, in conventional nucleic acid purification methods, selectivity to nucleic acids and proteins by electrostatic attraction forces is poor and the recovery yield of nucleic acids is low. Thus, a method of efficiently purifying nucleic acids rapidly, in which selectively binds nucleic acids and proteins and a high recovery yield of nucleic acids is achieved, is required.

Thus, the inventors of the present invention discovered that selectivity to nucleic acids and proteins is increased by using hydrogen bonding and nucleic acids is rapidly collected on a binding surface with the use of an electric field, thereby facilitating elution of the bound nucleic acids.

### SUMMARY OF THE INVENTION

The present invention provides a method of purifying nucleic acids using hydrogen bonding and an electric field, which has increased selectivity to nucleic acids and proteins and can purify nucleic acids rapidly.

According to an aspect of the present invention, there is provided a method of purifying nucleic acids using hydrogen bonding and an electric field, the method including: bringing a sample containing target nucleic acids into contact with an electrode coated with a material capable of forming hydrogen bonds with the target nucleic acids; applying a positive voltage to the electrode to move the target nucleic acids to the electrode so as to form hydrogen bonds between the target nucleic acids and the material on the electrode; washing the electrode; and applying a negative voltage to the electrode to elute the bound target nucleic acids.

### BRIEF DESCRIPTION OF THE DRAWINGS

The above and other features and advantages of the present invention will become more apparent by describing in detail exemplary embodiments thereof with reference to the attached drawings in which:

FIG. 1 is a schematic diagram illustrating an embodiment of the nucleic acid purification method according to the present invention;

FIG. 2 illustrates absorbance of samples at 550 nm;

FIG. 3 illustrates a crossing point (Cp) of samples in PCR; and

FIG. 4 illustrates relative fluorescence intensity of samples with proteins bound thereto.

### DETAILED DESCRIPTION OF THE INVENTION

The present invention relates to a method of purifying nucleic acids using hydrogen bonding and an electric field. Specifically, selectivity to nucleic acids and proteins is increased by using hydrogen bonding, a positive voltage is applied to more rapidly collect nucleic acids on a binding surface than movement by diffusion, and a negative voltage is applied to facilitate elution of the bound nucleic acids due to a repulsive force, thereby increasing the recovery yield of nucleic acids.

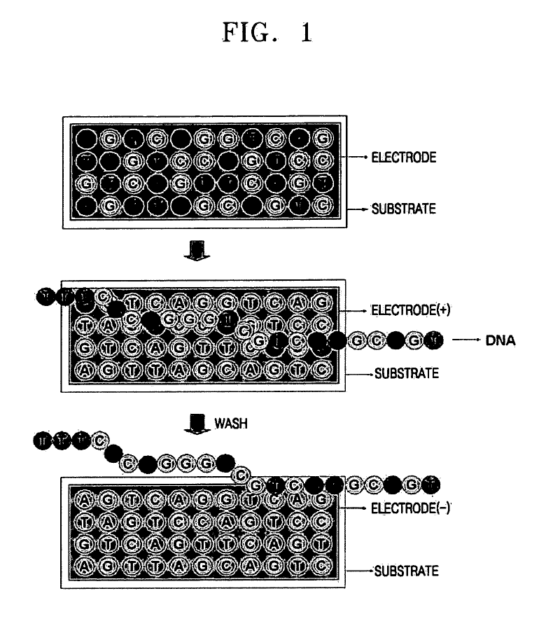

FIG. 1 is a schematic diagram illustrating an embodiment of the method of purifying nucleic acids according to the present invention. When a sample containing target nucleic acids to be purified is brought into contact with an electrode coated with a material capable of forming hydrogen bonds with the target nucleic acids, hydrogen bonds are formed.

Generally, hydrogen bonding occur between a hydrogen atom and a larger, adjacent non-metal, typically with a high electronegativity, such as O, N or F which will be denoted by X. Thus, a molecule as Y—H . . . X or even X—H . . . X as in the case of water, the electron of the H are pulled to the X due the high electronegativity. As the electron spends most of the time closer to X or Y, a positive charge is generated on the H (proton), and the X or Y take on a negative charge. The positive charge of one molecule then attracts the negative charge of the adjacent molecule. This attraction is hydrogen bonding. As an example, water has two hydrogen atoms and a oxygen atom. The hydrogen of one molecule is attracted to the oxygen of the next molecule.

Hydrogen bonding has important effects on various materials. In the case of ice, large space is formed due to a netty structure generated by hydrogen bonds. For example, when melting ice, energy is needed to break the hydrogen bonds between the water molecules surrounding one water molecule.

Next, when a positive voltage is applied to the electrode, target nucleic acids in the sample move closer to the electrode due to electrostatic attraction force to increase the number of hydrogen bonds between the target nucleic acid and the material on the electrode. The nucleic acid has a phosphate group, and thus is negatively charged at a neutral pH. Thus, when a positive voltage is applied to the electrode coated with the material capable of forming hydrogen bonds with the target nucleic acids, target nucleic acids in the sample move closer to the electrode due to electrostatic attraction forces by an electric field. That is, when an electric field is not generated, the target nucleic acids move by diffusion, and thus the possibility of hydrogen bonding between the material on the electrode and the target nucleic acids is low. However, when an electric field is generated, the target nucleic acids move more rapidly due to electrostatic attraction forces, thereby increasing the possibility of hydrogen bonding.

Then, the electrode is washed. Substances (ex. complementary nucleic acids) bound relatively strongly to the material on the electrode are not easily dissociated compared to substances (ex. proteins) bound relatively weakly to the material on the electrode. Thus, substances which are relatively weakly bound or are not bound to the material can be removed using a washing buffer such as PBS. The washing conditions can be varied depending on the binding forces between the nucleic acids to be separated and the material coated on the electrode.

Subsequently, when a negative voltage is applied to the electrode having target nucleic acids bound thereto, the target nucleic acids are eluted from the electrode due to electrostatic repulsive forces. Since the target nucleic acid is negatively charged, it is separated from the electrode due to electrode repulsive force. Thus, target nucleic acids in the sample are purified by hydrogen bond and electric field.

In an embodiment of the present invention, the electrode may be selected from the group consisting of metal electrodes, such as Au, Pt, Ag, and the like. The electrode is coated with a material capable of forming hydrogen bonds with target nucleic acids. The material is covalently bound to the electrode and may be the respective monomers of nucleic acids, G, A, T and C, to which a spacer having a thiol group, for example, HS—(CH₂)₆, is bound, such as HS—(CH₂)₆-G, HS—(CH₂)₆-A, HS—(CH₂)₆-T or HS—(CH₂)₆—C.

In an embodiment of the present invention, the electrode may be deposited to a support selected from the group consisting of a silicon substrate, a silicon wafer, a gel and a bead. The electrode is deposited to a support and the support may have a 3-dimensional (3-D) surface, such as that of a gel or bead, as well as a 2-dimensional surface, such as that of a silicon substrate or silicon wafer. A support having a 3-D surface can purify more nucleic acids due to larger surface area than a support having a 2-D surface. The support is not particularly restricted as long as it can support the electrode.

In an embodiment of the present invention, the material may be selected from the group consisting of DNA monomers, RNA monomers, PNA monomers, nucleosides, bases of nucleic acids, triplex-forming oligonucleotides, and oligonucleotides. The material is not particularly restricted as long as it can form hydrogen bonds with the target nucleic acids. When the material coated on the electrode is a DNA monomer, a RNA monomer, a PNA monomer, a nucleoside or a base of a nucleic acid, hydrogen bonding with single strand target nucleic acid is possible. Thus, purification of RNA or single strand DNA is mainly carried out. This is because two complementary single strand nucleic acids form hydrogen bonds therebetween, and thus a double strand nucleic acid is formed.

However, in the case of triplex-forming oligonucleotide [Nucleic Acids Research, 2001, vol 29, No. 23, 4873-4880], the target nucleic acid can be either double stranded and single stranded. That is, since the triplex-forming oligonucleotide can form hydrogen bonds with double strand target nucleic acid, for example, double strand DNA or RNA can be used as the target nucleic acid. Even when the target nucleic acid is single stranded, the triplex-forming oligonucleotide can also form double strand nucleic acid through hydrogen bonding. Thus, the triplex-forming oligonucleotide can form hydrogen bonds with double strand or single strand DNA or RNA.

In an embodiment of the present invention, the material coated on the electrode may be a polynucleotide capable of being complementarily bound to the target nucleic acids. When the material is a monomer of a nucleic acid, it is randomly bound to the electrode, which is advantageous in the purification of nucleic acids having an arbitrary sequence from proteins. However, it is preferable that the material is a polynucleotide when nucleic acids having a specific sequence are separated. Monomers of a nucleic acid which are arbitrarily coated on the electrode can partially form hydrogen bonds with the target nucleic acids, but formation of continuous hydrogen bonds can be impossible. Thus, nucleic acids having a specific sequence can be separated using an oligonucleotide or polynucleotide which can continuously form hydrogen bonds with the target nucleic acids.

In an embodiment of the present invention, the voltage may be in the range of −5 to +5 V. When the voltage is less than −5 V, the target nucleic acids in the sample move too slowly toward the electrode. When the voltage is greater than +5 V, electrolysis of a solution is caused.

The present invention will now be described in greater detail with reference to the following examples. The following examples are for illustrative purposes only and are not intended to limit the scope of the invention.

### EXAMPLES

### Example 1

### Identification of the Effects of Nucleic Acid Purification Through Measurement of Absorbance

In this example, an oligonucleotide was purified using an Au electrode on a substrate on which 4 types of DNA monomers were coated. 4 types of DNA monomers, i.e. G, A, T and C, were coated on the Au electrode on the substrate by reacting each 5.0 μM of HS—(CH₂)₆-G, HS—(CH₂)₆-A, HS—(CH₂)₆-T and HS—(CH₂)₆—C with the Au electrode at room temperature for 2 hrs. 5.0 μM of Cy3 labeled oligonucleotide (SEQ ID No: 1) was used as the target nucleic acid. Next, the Au electrode coated with 4 types of DNA monomers was disposed on the bottom of a cylindrical tube and the sample containing the target nucleic acid was brought into contact with the Au electrode. Then, a positive voltage of 1V was applied for 5 min. Then, the Au electrode was three times washed with 200 μl of 1× PBS. An eluent was added on the Au electrode, a negative voltage of 1V was applied for 5 min to elute an oligonucleotide from the Au electrode. The eluent used was 200 μl of distilled water.

For the eluent, an absorbance at 550 nm was measured and the amount of the oligonucleotide purified was determined. The respective samples were prepared as follows: Sample 1 was prepared by bringing the target nucleic acid into contact with the Au electrode coated with DNA monomers and applying a voltage according to the method of the present invention; Sample 2 was prepared in the same manner as Sample 1, except that a voltage was not applied in order to identify the effects of the electric field; Sample 3 was prepared in the same manner as Sample 1, except that a voltage was not applied and the Au electrode coated with DNA monomers was substituted by an amine glass in order to identify the effects of hydrogen bonding, in which the amine groups bound to a glass surface electrostatically interact with the negatively-charged oligonucleotide, and thus the oligonucleotide was bound to the glass surface, which was different from the method of the present invention in which DNA monomers were bound to the oligonucleotide due to hydrogen bonding; and Sample 4 was prepared in the same manner as Sample 1, except that a voltage was not applied, the Au electrode coated with DNA monomers was substituted by a silica glass, a chaotropic salt (which was bound to the silca glass using a PB buffer contained in MinElute PCR purification kit and was washed with a PE buffer) was used, and elution is carried out using 200 μl of distilled water.

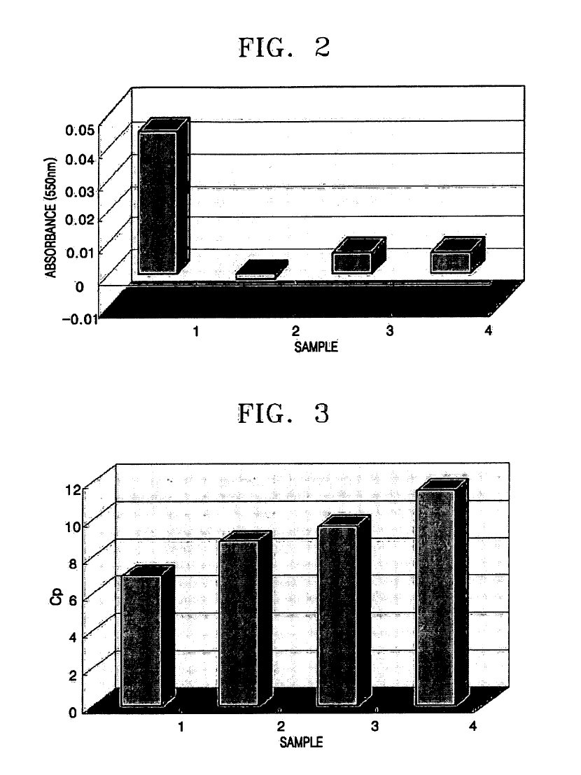

FIG. 2 illustrates the absorbance of each sample at 550 nm. Referring to FIG. 2, Sample 1, according to the method of the present invention in which a voltage is applied to the Au electrode coated with DNA monomers, has a higher absorbance than other three samples, which indicates that the recovery yield of the single stand DNA of Sample 1 is highest. That is, it can be seen that the target nucleic acid is more specifically purified by hydrogen bonding and an electric field.

### Example 2

### Identification of the Effects of Nucleic Acid Purification Through Polymerase Chain Reaction (PCR)

A sample was prepared in the same manner as in Example 1, except that 467 nM of a ssHBV DNA (SEQ ID No: 2) was used as the target nucleic acid. Then, PCR was performed from the respective DNA purified. The PCR primers used were as follows: a primer A (SEQ ID No: 3) and a primer B (SEQ ID No: 4). In the PCR, after a pre-denaturation at 95° C. for 1 min using Taq polymerase (Solgent, Korea), 50 cycles (denaturation at 95° C. for 5 sec, and annealing and extension at 62° C. for 15 sec) were performed. The amplified DNA was analyzed in Agilent 2100 BioAnalyzer (Agilent Technologies, Palo Alto, Calif.) with a commercially available DNA 500 assay sizing reagent sets.

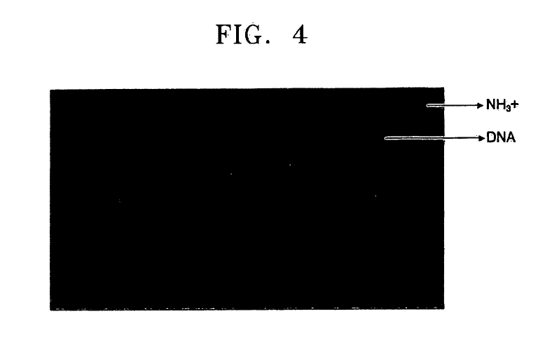

FIG. 3 illustrates the crossing point (Cp) of the respective samples in a PCR. Cp refers to the number of cycles at which the fluorescent signal is detected in a real-time PCR. That is, as the initial DNA concentration is higher, the fluorescent signal can be detected at a lower Cp. The Cp is also related to DNA purification. The higher DNA purity, the lower the Cp. Thus, it can be seen that as the Cp is lower, the DNA in the sample is a more specifically purified one.

As shown in FIG. 3, Sample 1 according to the method of the present invention has a lower Cp than other three samples, which indicates that the recovery yield of the single strand DNA of Sample 1 is highest. That is, it can be seen that the target nucleic acid is more specifically purified by hydrogen bond and electric field.

### Example 3

### Effects of Hydrogen Bonding on Selectivity to Nucleic Acids and Proteins

To identify whether the material capable of forming hydrogen bonds with the target nucleic acid has a binding specificity to nucleic acids, i.e., selectivity to nucleic acids and proteins, binding between the material and proteins was investigated. In the experiment, 1.4 μM DNA was spotted on an amine glass, 100 μg/mL of IgG-Cy3 was added thereto and was incubated for 1 hr. Then, the resultant was washed with 500 mL of a mixture of 1× PBS and 0.5% Tween 20. The emitted fluorescence was detected using an Exon scanner (Genepix 4000B).

FIG. 4 illustrates the relative intensity of fluorescence of the respective samples. In FIG. 4, “NH₃⁺” denotes no spotting of DNA and “DNA” denotes the spotting of DNA. As a result, the relative intensity of fluorescence is 10380±285 at no spotting of DNA and is 7084±306 at spotting of DNA, which indicates that the former has a higher intensity of fluorescence than the latter. It can be seen that since proteins more weakly bind to the DNA surface than to the NH₃⁺ surface, the relative intensity of fluorescence at the spotting of DNA is low. It is interpreted that the results are induced from a positively-charged NH₃⁺ having stronger electrostatic attraction forces to proteins compared to the negatively-charged DNA. While the material capable of forming hydrogen bonds with the target nucleic acid has weak binding forces to proteins, it has relatively strong binding forces to the target nucleic acid due to hydrogen bonding, thereby having selectivity to nucleic acids and proteins as the target material. In addition, since proteins are not easily bound to the material, the possibility of binding of the target nucleic acid increases. Thus, according to the method of the present invention, nucleic acids can be purified more efficiently.

As described above, according to the present invention, selectivity to nucleic acids and proteins increases due to hydrogen bonding, rapid nucleic acid purification is possible through an electric field, and the bound nucleic acids can be efficiently eluted.

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. A method of purifying nucleic acids using hydrogen bonding and an electric field, comprising:
bringing a sample containing target nucleic acids into contact with an electrode coated with a material capable of forming hydrogen bonds with the target nucleic acids;
applying to the electrode a positive voltage to move the target nucleic acids to the electrode so as to form hydrogen bonds with the material on the electrode;
washing the electrode; and
applying to the electrode a negative voltage to elute the bound target nucleic acids.

2. The method of claim 1, wherein the electrode is selected from the group consisting of Au, Pt and Ag.

3. The method of claim 1, wherein the electrode is deposited to a support selected from the group consisting of a silicon substrate, a silicon wafer, a gel, and a bead.

4. The method of claim 1, wherein the material is selected from the group consisting of DNA monomers, RNA monomers, PNA monomers, nucleosides, bases of nucleic acids, triplex-forming oligonucleotides, and oligonucleotides.

5. The method of claim 4, wherein the material is selected from the group consisting of HS—(CH₂)₆-G, HS—(CH₂)₆-A, HS—(CH₂)₆-T or HS—(CH₂)6—C.

6. The method of claim 1, wherein the target nucleic acid is a double strand or single strand nucleic acid.

7. The method of claim 6, wherein the target nucleic acid is RNA.

8. The method of claim 1, wherein the material is a polynucleotide capable of being complementarily bound to the target nucleic acid.

9. The method of claim 1, wherein the voltage is in the range of −5 V to +5 V.
